SEQUENCE LISTING (61 sequences)

SEQ ID NO: 1 - protein, 5 aa, Artificial Sequence
  Synthetic peptide
SYYWS

SEQ ID NO: 2 - protein, 16 aa, Artificial Sequence
  Synthetic peptide
YVYYSGTTNYNPSLKS

SEQ ID NO: 3 - protein, 10 aa, Artificial Sequence
  Synthetic peptide
IAVTGFYFDY

SEQ ID NO: 4 - protein, 12 aa, Artificial Sequence
  Synthetic peptide
RASQRVNNNYLA

SEQ ID NO: 5 - protein, 7 aa, Artificial Sequence
  Synthetic peptide
GASSRAT

SEQ ID NO: 6 - protein, 9 aa, Artificial Sequence
  Synthetic peptide
QQYDRSPLT

SEQ ID NO: 7 - protein, 118 aa, Artificial Sequence
  Synthetic peptide
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSS

SEQ ID NO: 8 - protein, 108 aa, Artificial Sequence
  Synthetic peptide
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIK

SEQ ID NO: 9 - protein, 241 aa, Artificial Sequence
  Synthetic peptide
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIK

SEQ ID NO: 10 - protein, 496 aa, Artificial Sequence
  Synthetic peptide
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 11 - protein, 118 aa, Artificial Sequence
  Synthetic peptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 12 - protein, 108 aa, Artificial Sequence
  Synthetic peptide
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 13 - protein, 241 aa, Artificial Sequence
  Synthetic peptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 14 - protein, 496 aa, Artificial Sequence
  Synthetic peptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 15 - protein, 14 aa, Artificial Sequence
  Synthetic peptide
GSSTGAVTSGNYPN

SEQ ID NO: 16 - protein, 7 aa, Artificial Sequence
  Synthetic peptide
GTKFLAP

SEQ ID NO: 17 - protein, 9 aa, Artificial Sequence
  Synthetic peptide
VLWYSNRWV

SEQ ID NO: 18 - protein, 5 aa, Artificial Sequence
  Synthetic peptide
KYAMN

SEQ ID NO: 19 - protein, 19 aa, Artificial Sequence
  Synthetic peptide
RIRSKYNNYATYYADSVKD

SEQ ID NO: 20 - protein, 14 aa, Artificial Sequence
  Synthetic peptide
HGNFGNSYISYWAY

SEQ ID NO: 21 - protein, 125 aa, Artificial Sequence
  Synthetic peptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 22 - protein, 109 aa, Artificial Sequence
  Synthetic peptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 23 - protein, 249 aa, Artificial Sequence
  Synthetic peptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 24 - protein, 984 aa, Artificial Sequence
  Synthetic peptide
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 982 aa, Artificial Sequence
  Synthetic peptide
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 984 aa, Artificial Sequence
  Synthetic peptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 982 aa, Artificial Sequence
  Synthetic peptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 29 - protein, 40 aa, Homo sapiens
SGVTCADGPCFNGGLCVGGADPDSAYICHCPPGFQGSNCE

SEQ ID NO: 30 - protein, 37 aa, Homo sapiens
RVDRCSLQPCRNGGLCLDLGHALRCRCRAGFAGPRCE

SEQ ID NO: 31 - protein, 78 aa, Homo sapiens
SGVTCADGPCFNGGLCVGGADPDSAYICHCPPGFQGSNCEKRVDRCSLQPCRNGGLCLDLGHALRCRCRAGFAGPRCE

SEQ ID NO: 32 - protein, 5 aa, Artificial Sequence
  Synthetic peptide
SYDMS

SEQ ID NO: 33 - protein, 17 aa, Artificial Sequence
  Synthetic peptide
LISGGGSQTYYAESVKG

SEQ ID NO: 34 - protein, 11 aa, Artificial Sequence
  Synthetic peptide
PSGHYFYAMDV

SEQ ID NO: 35 - protein, 11 aa, Artificial Sequence
  Synthetic peptide
RASQGISNWLA

SEQ ID NO: 36 - protein, 7 aa, Artificial Sequence
  Synthetic peptide
AASSLQS

SEQ ID NO: 37 - protein, 9 aa, Artificial Sequence
  Synthetic peptide
QQAESFPHT

SEQ ID NO: 38 - protein, 120 aa, Artificial Sequence
  Synthetic peptide
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSS

SEQ ID NO: 39 - protein, 107 aa, Artificial Sequence
  Synthetic peptide
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIK

SEQ ID NO: 40 - protein, 450 aa, Artificial Sequence
  Synthetic peptide
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 213 aa, Artificial Sequence
  Synthetic peptide
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 42 - protein, 4 aa, Artificial Sequence
  Synthetic peptide
GGGG

SEQ ID NO: 43 - protein, 5 aa, Artificial Sequence
  Synthetic peptide
GGGGS

SEQ ID NO: 44 - protein, 5 aa, Artificial Sequence
  Synthetic peptide
GGGGQ

SEQ ID NO: 45 - protein, 6 aa, Artificial Sequence
  Synthetic peptide
SGGGGS

SEQ ID NO: 46 - protein, 6 aa, Artificial Sequence
  Synthetic peptide
PGGGGS

SEQ ID NO: 47 - protein, 6 aa, Artificial Sequence
  Synthetic peptide
PGGDGS

SEQ ID NO: 48 - protein, 9 aa, Artificial Sequence
  Synthetic peptide
GGGGSGGGS

SEQ ID NO: 49 - protein, 10 aa, Artificial Sequence
  Synthetic peptide
GGGGSGGGGS

SEQ ID NO: 50 - protein, 15 aa, Artificial Sequence
  Synthetic peptide
GGGGSGGGGSGGGGS

SEQ ID NO: 51 - protein, 484 aa, Artificial Sequence
  Synthetic peptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 480 aa, Artificial Sequence
  Synthetic peptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 53 - protein, 484 aa, Artificial Sequence
  Synthetic peptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 480 aa, Artificial Sequence
  Synthetic peptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 55 - protein, 484 aa, Artificial Sequence
  Synthetic peptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 480 aa, Artificial Sequence
  Synthetic peptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 57 - protein, 484 aa, Artificial Sequence
  Synthetic peptide
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 480 aa, Artificial Sequence
  Synthetic peptide
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 59 - protein, 981 aa, Artificial Sequence
  Synthetic peptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 268 aa, Artificial Sequence
  Synthetic peptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 61 - protein, 288 aa, Artificial Sequence
  Synthetic peptide
MQIPQAPWPVVWAVLQLGWRPGWFLESPDRPWNAPTFSPALLLVTEGDNATFTCSFSNASESFVLNWYRMSPSNQTDKLAAFPEDRSQPGQDCRFRVTRLPNGRDFHMSVVRARRNDSGTYLCGAISLAPKAQIKESLRAELRVTERRAEVPTAHPSPSPRPAGQFQALVVGVVGGLLGSLVLLVWVLAVICSRAAQGTIEARRTGQPLKEDPSAVPVFSVDYGELDFQWREKTPEPPAPCVPEQTEYATIVFPSGLGTSSPARRGSADGPRSPRPLRPEDGHCSWPL

(19) United States
(12) Patent Application Publication
(10) Pub. No.: US 2024/0317856 A1
(43) Pub. Date: Sep. 26, 2024

(54) DOSING REGIMEN FOR COMBINATION THERAPY TARGETING DLL3 AND PD-1

(71) Applicant: AMGEN INC., Thousand Oaks, CA (US)

(72) Inventors: Nooshin Hashemi Sadraei, Thousand Oaks, CA (US); Aditya Shetty, Thousand Oaks, CA (US); Mukul Minocha, Thousand Oaks, CA (US); Marie-Anne Damiette Smit, Thousand Oaks, CA (US); Hansen Wong, Thousand Oaks, CA (US); Siddhartha Roychoudhury, Thousand Oaks, CA (US)

(21) Appl. No.: 18/289,317

(22) PCT Filed: May 6, 2022
(86) PCT No.: PCT/US22/28135

§ 371 Date: Nov. 2, 2023

Related U.S. Application Data
(60) Provisional application No. 63/186,569, filed on May 10, 2021.

(51) Int. Cl. C07K 16/28; A61K 39/00

(52) U.S. Cl. CPC C07K 16/2809; C07K 16/2818; A61K 2039/507; A61K 2039/545; C07K 2317/21; C07K 2317/31; C07K 2317/52; C07K 2317/622; C07K 2317/76

(57) ABSTRACT

The present invention provides a method for the treatment of DLL3-positive cancer or SCLC, comprising administering to a subject in need thereof an anti-DLL3 agent and an anti-PD-1 antibody. Step dosing of the anti-DLL3 agent is also disclosed.

### PRIORITY

This application is a U.S. national stage of International Application No. PCT/US2022/028135, filed on May 6, 2022, which claims benefit to U.S. Provisional Application No. 63/186,569, filed May 10, 2021, the contents of which are hereby incorporated by reference in their entirety.

### SUBMISSION OF SEQUENCE LISTING ON ASCII TEXT FILE

The content of the following submission on ASCII text files is incorporated herein by reference in its entirety: a computer readable form (CRF) of the Sequence Listing (file name: A-2789-WO01-SEC_Sequence Listing_ST25, date created May 6, 2022, size: 123,163 byes).

### FIELD OF THE INVENTION

The present application relates to dosage and administration of combination therapy targeting DLL3 and PD-1 for the treatment of cancer.

### BACKGROUND OF THE INVENTION

Delta-like 3 (DLL3) is a type 1 transmembrane protein and noncanonical Notch ligand. DLL3 is a promising target for the development of T-cell therapies due to its high expression on the cell surface of neuroendocrine tumors, and minimal, mainly cytoplasmic localization in normal tissues (Owen et al., J Hematol Oncol., 12:61 (2019)). Small cell lung cancer (SCLC) is a neuroendocrine cancer wherein DLL3 is differentially expressed. Using immunohistochemistry (IHC), 85% of SCLC tumors stained positive for DLL3 in a pattern consistent with both membranous and cytoplasmic expression. In contrast, low levels of DLL3 protein expression were detected in normal brain, pancreatic islets, and pituitary gland with a cytoplasmic staining pattern (Saunders et al, Sci Transl Med. 7:302ra136 (2015)).

SCLC is an aggressive form of lung cancer with a poor prognosis and limited therapeutic options and represents about 10-15% of lung cancers. Survival rates have remained low for several decades, with only 5% of SCLC patients surviving five years, in a large part due to the lack of new therapies to combat this form of lung cancer. SCLC is characterized by neuroendocrine differentiation, a high growth fraction, rapid doubling time and early establishment of widespread metastatic lesions. About a third of patients present with limited stage disease. Most patients present with extensive-stage disease, defined by the presence of tumors in only one side of the chest and that fit in a single radiation field. These stages impact available therapeutic regiments, with limited stage disease treated with chemotherapy and radiation and extensive stage disease treated with chemotherapy alone.

Patients with SCLC typically respond well to the current front-line therapy, which includes etoposide and cisplatin, but invariably quickly relapse with chemoresistant disease, for which no therapeutic options are currently available. Prognosis in the relapsed refractory setting is extremely poor, with rapid disease progression and short median survival of less than six months. Patients with extended disease SCLC develop drug resistance and die as a result of disease at a median time of 10 to 12 months from diagnosis.

AMG 757 is a bispecific T-cell engager (BiTE®) molecule targeting DLL3 on cancer cells and CD3 on T-cells. It is developed for the treatment of neuroendocrine cancers such as SCLC. AMG 757 is being evaluated in a clinical trial for treating SCLC.

Pembrolizumab (Keytruda®) and nivolumab (Opdivo®) are antibodies against programmed cell death-1 (PD-1). Both have been approved in the US for the treatment of patients with metastatic SCLC who have progression after platinum-based chemotherapy and at least 1 other line of therapy. However, the approvals were based on relatively low response rates (19% with pembrolizumab and 12% with nivolumab). Studies evaluating nivolumab as second-line or maintenance therapy have failed to meet their primary endpoints (Reck et al., Annals of Oncology. 29:x39-x43 (2018)).

There is an unmet medical need for the development of therapies for the treatment of SCLC.

### SUMMARY OF THE INVENTION

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).
E1: A method of treating DLL3-positive cancer, comprising administering to a subject in need thereof an anti-DLL3 agent and an anti-PD-1 antibody, wherein the anti-DLL3 agent is administered at a dose of from 0.3 mg to 30 mg or from 3 mg to 100 mg once every two weeks, and wherein the anti-PD-1 antibody is administered at a dose of 480 mg once every four weeks.
E2: A method of treating DLL3-positive cancer, comprising administering to a subject in need thereof an anti-DLL3 agent and an anti-PD-1 antibody, wherein the anti-DLL3 agent is administered in a 28-day cycle according to the following schedule: a) a first dose of from 0.3 or 1 mg on cycle 1 day 1, b) a second dose on cycle 1 day 8, and c) a third dose on cycle 1 day 15, and e) one or more subsequent doses starting on cycle 2 day 1 and once every two weeks thereafter, and wherein the second, third, and subsequent doses are the same, are each from 0.3 mg to 30 mg or from 3 mg to 100 mg, and are higher than the first dose, and wherein the anti-PD-1 antibody is administered at a dose of 480 mg once every four weeks.
E3: The method of E1 or E2, wherein the anti-DLL3 positive cancer is small cell lung cancer (SCLC).
E4: The method of any one of E1-E3, wherein the anti-DLL3 positive cancer is Relapsed/refractory (RR) SCLC or Extensive disease (ED) SCLC.
E5: The method of any one of E1-E4, wherein the anti-DLL3 agent is a bispecific T cell engaging antigen-binding polypeptide comprising two binding domains: the first domain binds to human DLL3, and the second domain binds to human CD3.
E6: The method of E4, wherein the DLL3-binding domain binds to an epitope of human DLL3 comprised within the amino acid sequence of SEQ ID NO:29.
E7: The method of E5 or E6, wherein the DLL3-binding domain comprises (a) a heavy chain variable region (VH) that comprises: (i) a VH complementarity determining region one (CDR-H1) comprising the amino acid sequence of SEQ ID NO:1; (ii) a CDR-H2 comprising the amino acid sequence of SEQ ID NO:2; and (iii) a CDR-H3 comprising the amino acid sequence of SEQ ID NO:3; and (b) a light chain variable region (VL) that comprises: (i) a VL complementarity determining region one (CDR-L1) comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6.
E8: The method of any one of E5-E7, wherein the DLL3-binding domain comprises: (1) a VH that comprises the amino acid sequence of SEQ ID NO:7, and a VL that comprises the amino acid sequence of SEQ ID NO:8, or (2) a VH that comprises the amino acid sequence of SEQ ID NO:11, and a VL that comprises the amino acid sequence of SEQ ID NO:12.
E9: The method of any one of ES-E8, wherein the VH and VL of the DLL3-binding domain are joined by a linker to form a single chain Fv (scFv).
E10: The method of E9, wherein the linker comprises a sequence selected from any one of SEQ ID NOs:42-50.
E11: The method of E9 or E10, wherein the linker comprises (Gly4Ser)x, where x is an integer of 1 or greater (e.g. 1, 2, 3 or 4).
E12: The method of any one of E5-E11, wherein the DLL3-binding domain comprises the amino acid sequence of SEQ ID NO:9 or SEQ ID NO:13.
E13: The method of any one of E5-E12, wherein the CD3-binding domain comprises: (a) a VH that comprises: a CDR-H1 comprising the amino acid sequence of SEQ ID NO: 18, a CDR-H2 comprising the amino acid sequence of SEQ ID NO: 19, and a CDR-H3 comprising the amino acid sequence of SEQ ID NO:20; and a VL that comprises: a CDR-L1 comprising the amino acid sequence of SEQ ID NO:15, a CDR-L2 comprising the amino acid sequence of SEQ ID NO:16, and a CDR-L3 comprising the amino acid sequence of SEQ ID NO:17.
E14: The method of any one of E5-E13, wherein the CD3-binding domain comprises: a VH that comprises the amino acid sequence of SEQ ID NO:21, and a VL that comprises the amino acid sequence of SEQ ID NO:22.
E15: The method of E13 or E14, wherein the VH and VL of the CD3-binding domain are joined by a linker to form a single chain Fv (scFv).
E16: The method of E15, wherein the linker comprises a sequence selected from any one of SEQ ID NOs:42-50.
E17: The method of E15 or E16, wherein the linker comprises (Gly4Ser)x, where x is an integer of 1 or greater (e.g. 1, 2, 3 or 4).
E18: The method of any one of E13-E17, wherein the CD3-binding domain comprises the amino acid sequence of SEQ ID NO:23.
E19: The method of any one of E5-E18, wherein the DLL3-binding domain and the CD3-binding domain are joined by a linker.
E20: The method of E19, wherein the linker is a peptide linker comprising a sequence selected from any one of SEQ ID NOs:42-50.
E21: The method of E19 or E20, wherein the linker is a peptide linker comprises (Gly4Ser)x, where x is an integer of 1 or greater (e.g., 1, 2, 3 or 4).
E22: The method of any one of E5-E21, the anti-DLL3 agent is a bispecific T cell engaging antigen-binding polypeptide comprising a DLL3-binding domain and a CD3-binding domain. The DLL3-binding domain comprises (a) a heavy chain variable region (VH) that comprises: (i) a VH complementarity determining region one (CDR-H1) comprising the amino acid sequence of SEQ ID NO:1; (ii) a CDR-H2 comprising the amino acid sequence of SEQ ID NO:2; and (iii) a CDR-H3 comprising the amino acid sequence of SEQ ID NO:3; and (b) a light chain variable region (VL) that comprises: (i) a VL complementarity determining region one (CDR-L1) comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6. The CD3-binding domain comprises (a) a VH that comprises: (i) a CDR-H1 comprising the amino acid sequence of SEQ ID NO:18, (ii) a CDR-H2 comprising the amino acid sequence of SEQ ID NO:19, and (iii) a CDR-H3 comprising the amino acid sequence of SEQ ID NO:20; and (b) a VL that comprises: (i) a CDR-L1 comprising the amino acid sequence of SEQ ID NO:15, (ii) a CDR-L2 comprising the amino acid sequence of SEQ ID NO: 16, and (iii) a CDR-L3 comprising the amino acid sequence of SEQ ID NO:17.
E23: The method of any one of E5-E22, the DLL3-binding domain comprises a VH that comprises the amino acid sequence of SEQ ID NO:7, and a VL that comprises the amino acid sequence of SEQ ID NO:8, and the CD3-binding domain comprises a VH that comprises the amino acid sequence of SEQ ID NO:21, and a VL that comprises the amino acid sequence of SEQ ID NO:22.
E24: The method of any one of E5-E22, the DLL3-binding domain comprises a VH that comprises the amino acid sequence of SEQ ID NO:11, and a VL that comprises the amino acid sequence of SEQ ID NO:12, and the CD3-binding domain comprises a VH that comprises the amino acid sequence of SEQ ID NO:21, and a VL that comprises the amino acid sequence of SEQ ID NO:22.
E25: The method of any one of E5-E23, wherein the DLL3-binding domain comprises the amino acid of SEQ ID NO:9 and the CD3-binding domain comprises the amino acid of SEQ ID NO: 23.
E26: The method of any one of E5-E21 or E24, the DLL3-binding domain comprises the amino acid of SEQ ID NO: 13 and the CD3-binding domain comprises the amino acid of SEQ ID NO:23.
E27: The method of E25, wherein the anti-DLL3 agent comprises the amino acid sequence of SEQ ID NO:10.
E28: The method of E27, wherein the anti-DLL3 agent comprises the amino acid sequence of SEQ ID NO:14.
E29: The method of any one of E5-E28, wherein the anti-DLL3 agent further comprises a third domain that extends or enhance the serum half-life of the anti-DLL3 agent.
E30: The method of E29, wherein the third domain comprises the amino acid sequence selected from any one of SEQ ID NOs:51-58.
E31: The method of any one of E5-E22, E24, E26, E28-E30, wherein the anti-DLL3 agent comprises the amino acid of SEQ ID NO:27 or 59.
E32: The method of any one of E5-E31, wherein the anti-PD-1 antibody comprises (a) a VH that comprises: (i) a CDR-H1 comprising the amino acid sequence of SEQ ID NO: 32, (ii) a CDR-H2 comprising the amino acid sequence of SEQ ID NO: 33, (iii) a CDR-H3 comprising the amino acid sequence of SEQ ID NO: 34; and (b) a VL that comprises: (i) a CDR-L1 comprising the amino acid sequence of SEQ ID NO: 35, (ii) a CDR-L2 comprising the amino acid sequence of SEQ ID NO: 36, (iii) a CDR-L3 comprising the amino acid sequence of SEQ ID NO: 37.
E33: The method of any one of E5-E32, wherein the anti-PD-1 antibody comprises a VH that comprises the amino acid sequence of SEQ ID NO: 38, and a VL that comprises the amino acid sequence of SEQ ID NO: 39.
E34: The method of any one of E5-E33, wherein the anti-PD-1 antibody comprises a heavy chain (HC) that comprises the amino acid sequence of EQ ID NO:40, and a light chain (LC) that comprises the amino acid sequence of SEQ ID NO:41.
E35: The method of any one of E1 or E3-E34, wherein the anti-DLL3 agent is administered once every two weeks at a dose of: from about 0.3 mg to about 30 mg, from about 1 mg to about 30 mg, from about 3 mg to about 30 mg or from about 10 mg to about 30 mg.
E36: The method of E35, wherein the anti-DLL3 agent is administered once every two weeks at a dose of about 0.3 mg, 1 mg, 3 mg, 10 mg, 25 mg or 30 mg.
E37: The method of any one of E1 or E3-E34, wherein the anti-DLL3 agent is administered once every two weeks at a dose of: from about 3 mg to about 100 mg, from about 10 mg to about 100 mg, or from about 30 mg to about 100 mg.
E38: The method of E37, wherein the anti-DLL3 agent is administered once every two weeks at a dose of about 3 mg, 10 mg, 25 mg, 30 mg, 50 mg, 75 mg or 100 mg.
E39: The method of any one of E2-E34, wherein the second, third, and subsequent doses of the anti-DLL3 agent are each from about 0.3 mg to about 30 mg, from about 1 mg to about 30 mg, from about 3 mg to about 30 mg or from about 10 mg to about 30 mg.
E40: The method of any one of E39, wherein the second, third, and subsequent doses of the anti-DLL3 agent are each at a dose of about 0.3 mg, 1 mg. 3 mg, 10 mg, 25 mg or 30 mg.
E41: The method of any one of E2-E34, wherein the second, third, and subsequent doses of the anti-DLL3 agent are each from about 3 mg to about 100 mg, from about 10 mg to about 100 mg, or from about 30 mg to about 100 mg.
E42: The method of E41, wherein the second, third, and subsequent doses of the anti-DLL3 agent are each a dose of about 3 mg, 10 mg. 25 mg, 30 mg, 50 mg, 75 mg or 100 mg.
E43: The method of any one of E1 or E3-E38, wherein the anti-DLL3 agent is administered on day 1 and day 15 of a 28-day cycle and the anti-PD-1 antibody is administered on day 1, day 8 or day 15 of a 28-day cycle.
E44: The method of E43, wherein the anti-PD-1 antibody is administered on day 1, day 8 or day 15 in cycle one of a 28-day cycle, and then on day 1 or day 15 starting in cycle two and thereafter.
E45: The method of any one of E2-E34 or E39-E42, wherein the anti-PD-1 antibody is administered on day 1, day 8 or day 15 in cycle 1 of a 28-day cycle, and then on day 1 or day 15 starting in cycle two and thereafter.
E46: The method of E44 or E45, wherein a) if the anti-PD-1 antibody is administered on day 1 or day 8 in cycle one, then the antibody is administered on day 1 starting in cycle two and thereafter, or b) if the anti-PD-1 antibody is administered on day 15 in cycle one, then the antibody is administered on day 15 in cycle 2 and thereafter.
E47: The method of any one of E1-E46, wherein the method further comprises administering one or more additional therapeutic agent to the subject.
E48: The method of E47, wherein the one or more additional therapeutic agents is a corticosteroid (e.g., dexamethasone), saline, or tocilizumab.
E49: The method of any one of E47 or E48, wherein the one or more additional therapeutic agent is administered to the subject in the first cycle wherein the anti-DLL3 agent is administered.
E50: The method of any one of E1-E50, wherein the anti-DLL3 agent is prepared by a process wherein a host cell comprising a nucleic acid encoding the anti-DLL3 agent described in any one of E5-E31 is cultured under conditions allowing the expression of the anti-DLL3 agent and the expressed anti-DLL3 agent is then recovered from the cell culture, and wherein the anti-PD-1 antibody is prepared by a process wherein a host cell comprising a nucleic acid encoding the anti-PD-1 antibody described in any one of E32-E34 is cultured under conditions allowing the expression of the antibody and the expressed anti-PD-1 antibody is then recovered from the cell culture
E51: The method of any one of E1-E50, wherein the subject is a human.
E52: An anti-DLL3 agent and an anti-PD-1 antibody for use in a method as set forth in any one of embodiments E1-E51.
E53: An anti-DLL3 agent and an anti-PD-1 antibody for use in the treatment of DLL3-positive cancer (e.g., SCLC), wherein the anti-DLL3 agent and the anti-PD-1 antibody are administered as set forth in any one of embodiments E1-E51.
E54: Use of an anti-DLL3 agent and an anti-PD-1 antibody for the manufacture of a medicament for the treatment of SCLC, wherein the medicament is prepared to be administered as set forth in any one of embodiments E1-E51.
E55: Use of an anti-DLL3 agent and an anti-PD-1 antibody in the preparation of a medicament for the treatment of an DLL3-positive cancer, wherein the anti-DLL3 agent and the anti-PD-1 antibody are administered as set for in any one of embodiments E1-E51.

### BRIEF DESCRIPTION OF THE DRAWINGS

FIG. 1 shows AMG 757 and AMG 404 dose levels in the clinical study exemplified in Example 2.

### DETAILED DESCRIPTION

AMG 757 is a half-life-extended BiTE® (bispecific T cell engager) molecule developed for the treatment of SCLC. The activity of AMG 757 requires the simultaneous binding to both target cells (DLL3⁺ cells) and T cells. The pharmacological effect of AMG 757 is mediated by specific redirection of previously primed cytotoxic CD8⁺ or CD4⁺ T lymphocytes to kill DLL3⁺ cells. AMG 757 is being evaluated in a first-in-human study in subjects with SCLC (Study 20160323) and was found to have anti-tumor activity starting at dose level of 0.3 mg once every two weeks (Q2W) and with acceptable safety at doses up to 100 mg Q2W.

AMG 404 is a fully human antibody that binds human PD-1 with high affinity and blocks the ability of this receptor to interact with its ligands, programmed death-ligand 1 (PD-L1) and programmed death-ligand 2 (PD-L2). AMG 404 is being evaluated in a phase 1 study (Study 20180143) of subjects with solid tumors and was found to be effective against solid tumors.

A number of clinical trials were initiated recently for the treatment of small cell lung cancer using PD1/PDL1 inhibitors in combination with other anti-cancer agents. See e.g., NCT04702880 and NCT04256421 (SKYSCRAPER-02). However, recent announcement of failure of the SKYSCRAPER-02 trial highlights the uncertainty of combining PD1/PDL1 inhibitors with other anti-cancer agents for the treatment of this difficult to treat cancer. There remains a high unmet need and ongoing challenges in targeting this tumor type.

The combination of AMG 757 and anti-PD-1 antibodies increases T-cell mediated redirected lysis of tumor cells that express DLL3 compared to AMG 757 alone (Amgen Study Report R20190104). It is believed that upregulation of PD1/PDL1 in the tumor microenvironment is a mechanism of resistance to BiTE therapy that treatment with anti-PD1 therapy may mitigate.

As disclosed and exemplified herein, a Phase 1 clinical study was conducted for the treatment of SCLC, using agents that target DLL3 (e.g., AMG 757) and PD-1 (e.g., pembrolizumab or AMG 404).

### 1. Definition

Some of exemplary bispecific anti-DLL3 agents disclosed herein (such as BiTE® molecules) are bispecific T cell engaging antigen-binding polypeptides. These polypeptides are recombinant proteins comprising two binding domains, each domain derived from an antigen-binding fragment of a full-length antibody. Such antigen-binding fragment retains the ability to specifically bind to an antigen (preferably with substantially the same binding affinity). Examples of an antigen-binding fragment includes (i) a Fab fragment, a monovalent fragment consisting of the VL. VH. CL and CH1 domains; (ii) a F(ab′)₂ fragment, a bivalent fragment comprising two Fab fragments linked by a disulfide bridge at the hinge region: (iii) a Fd fragment consisting of the VH and CH¹ domains; (iv) a Fv fragment consisting of the VL and VH domains of a single arm of an antibody, and (v) a dAb fragment (Ward et al., 1989 Nature 341:544-546), which consists of a VH domain. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science 242:423-426 (1988) and Huston et al., 1988. Proc. Natl. Acad. Sci. USA 85:5879-5883.

A “variable domain” refers to the variable region of the antibody light chain (VL) or the variable region of the antibody heavy chain (VH), either alone or in combination. As known in the art, the variable regions of the heavy and light chains each consist of four framework regions (FR) connected by three complementarity determining regions (CDRs), and contribute to the formation of the antigen-binding site of antibodies.

The “Complementarity Determining Regions” (CDRs) of exemplary agents targeting DLL3 and PD-1 are provided in the Sequence Table. The CDRs can be defined according to Kabat, Chothia, the accumulation of both Kabat and Chothia, AbM, contact, North, and/or conformational definitions or any method of CDR determination well known in the art. See, e.g., Kabat et al., 1991, Sequences of Proteins of Immunological Interest, 5th ed. (hypervariable regions); Chothia et al., 1989. Nature 342:877-883 (structural loop structures). AbM definition of CDRs is a compromise between Kabat and Chothia and uses Oxford Molecular's AbM antibody modeling software (Accelrys®). The identity of the amino acid residues in a particular antibody that make up a CDR can be determined using methods well known in the art.

The term “treatment” includes prophylactic and/or therapeutic treatments. If it is administered prior to clinical manifestation of a condition, the treatment is considered prophylactic. Therapeutic treatment includes, e.g., ameliorating or reducing the severity of a disease, or shortening the length of the disease. Also, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the cancer. For example, the methods can treat cancer by virtue of enhancing the T cell activity or an immune response against the cancer, reducing tumor or cancer growth, reducing metastasis of tumor cells, increasing cell death of tumor or cancer cells, and the like. In exemplary aspects, the methods treat by way of delaying the onset or recurrence of the cancer by 1 day, 2 days, 4 days, 6 days, 8 days, 10 days, 15 days, 30 days, two months, 4 months, 6 months, 1 year, 2 years, 4 years, or more. In exemplary aspects, the methods treat by way increasing the survival of the subject. In various aspects, the treatment provided by the methods of the present disclosure provides a therapeutic response as per Response Evaluation Criteria in Solid Tumors (RECIST) or other like criteria. RECIST is a set of criteria to evaluate the progression, stabilization or responsiveness of tumors and/or cancer cells jointly created by the National Cancer Institute of the United States, the National Cancer Institute of Canada Clinical Trials Group and the European Organisation for Research and Treatment of Cancer. According to RECIST, certain tumors are measured in the beginning of an evaluation (e.g., a clinical trial), in order to provide a baseline for comparison after treatment with a drug. The response assessment and evaluation criteria for tumors are published in Eisenhauer et. al., Eur J Cancer 45:228-247 (2009) and Litière et. al., Journal of Clinical Oncology 37(13): 1102-1110 (2019) DOI: 10.1200/JCO.18.01100. In various instances, the treatment provided by the methods of the present disclosure provides a therapeutic response as per a modified RECIST tumor response assessment, as follows:

Summary of Measurement and Tumor Response
Assessment Based on Modified RECIST 1.1

| | |
|---|---|
| Measurable lesions | Non-nodal lesions: ≥10 mm (unidimensional |
| | measurement) |
| | Pathologic lymph nodes: longest diameter short |
| | axis ≥15 mm |
| Measurement | Non-nodal lesions: The longest diameter (mm) in |
| of each | the axial plane |
| lesion | Pathologic lymph nodes: short axis (mm) |
| Tumor burden | Sum of the longest diameters (SLD) of all index |
| | lesions |
| | Up to 5 lesions per organ, up to 10 total |
| Response assessment: | CR: Disappearance of all lesions |
| index lesions | Pathologic lymph nodes short axis <10 mm |
| (calculated from % | PR: ≥30% decrease from baseline |
| change in | SD: Does not meet criteria for CR, PR or |
| tumor burden) | progressive disease. |
| | Progressive disease: ≥20% increase (and ≥5 mm |
| | absolute increase) from nadir |
| Response | CR: Disappearance of all lesions |
| assessment: | Pathologic lymph nodes short axis <10 mm |
| non-index lesions | SD: Persistence of one or more non-index |
| | lesion(s) |
| | Progressive disease: Unequivocal progression of |
| | existing non-index lesions |
| New Lesions | The presence of new lesion(s) defines progression |
| Confirmation | Confirmation by subsequent assessment after ≥4 |
| | weeks required for CR, PR and progressive |
| | disease. |

Summary of Modified RECIST 1.1 Overall Response Assessment

| | | | Overall Response |
|---|---|---|---|
| Index lesions (tumor | | New | using modified |
| burden)^{a}, % | Non-Index lesions | lesions | RECIST 1.1 |
| ↓ 100% | Absent | Absent | CR^{b} |
| None^{d} | Absent | Absent | CR^{b} |
| ↓ 100% | Present | Absent | PR^{b} |
| ↓ ≥30% | Absent/Present | Absent | PR^{b} |
| ↓ <30% to ↑ <20% | Absent/Present | Absent | SD |
| None^{d} | Present | Absent | SD |
| ↑ ≥20% | Any | Any | Progressive disease^{c} |
| Any | Unequivocal | Any | Progressive disease^{c} |
| | progression | | |
| Any | Any | Present | Progressive disease^{b} |
| NA/ND/UE | Absent/Present | Absent | UE |
| None^{d} | NA/ND/UE | Absent | UE |

CR = complete response;
NA = not available;
ND = not done;
PR = partial response;
RECIST = Response Evaluation Criteria in Solid Tumors;
UE = unable to evaluate
^{a}Decrease assessed relative to baseline. Increase assessed relative to nadir.
^{b}Response: CR and PR require a confirmation assessment after ≥4 weeks, may also wait until the next scheduled imaging
^{c}Progression: Progressive disease requires a confirmation assessment 4 to 6 weeks after initial radiographic progressive disease is observed
^{d}Subjects with non-index lesions only

Accordingly, methods of slowing the progression of a DLL3-positive cancer in a subject, enhancing the T cell activity or an immune response against a DLL3-positive cancer in a subject, reducing growth of a DLL3-positive tumor or DLL3-positive cancer in a subject, reducing metastasis of DLL3-positive tumor cells in a subject, increasing cell death of DLL3-positive tumor or cancer cells in a subject, delaying the onset or recurrence of a DLL3-positive cancer in a subject and/or increasing the survival of a subject are provided herein. Also, a method of treating a DLL3-positive cancer to provide a complete response (CR), partial response (PR), or stable disease (SD), as per a modified RECIST 1.1, in a subject is provided. In various aspects, the method comprises administering to the subject an anti-DLL3 agent and an anti-PD-1 antibody in accordance with the present disclosures. For example, in various aspects, the method comprises administering an anti-DLL3 agent comprising the amino acid sequence of SEQ ID NOs: 13 and 23 and an anti-PD-1 antibody comprising the amino acid sequence of SEQ ID NOs: 38 and 39, wherein the anti-DLL3 agent is administered at a dose of from 0.3 mg to 30 mg or from 3 mg to 100 mg once every two weeks, and wherein the anti-PD-1 antibody is administered at a dose of 480 mg once every four weeks. In various instances, the anti-DLL3 agent is administered in a 28-day cycle according to the following schedule: a) a first dose of 0.3 mg or 1 mg on cycle 1 day 1, b) a second dose on cycle 1 day 8, c) a third dose on cycle 1 day 15, and d) one or more subsequence doses starting on cycle 2 day 1 and once every two weeks thereafter, wherein the second, third, and subsequent doses are the same, are each from 0.3 mg to 30 mg or from 3 mg to 100 mg, and are higher than the first dose, and wherein the anti-PD-1 antibody is administered at a dose of 480 mg once every four weeks. In various aspects, the method comprises administering an anti-DLL3 agent comprising the amino acid sequence of SEQ ID NOs: 13 and 23 and an anti-PD-1 antibody approved by a regulatory agency (e.g., the FDA or EMA), wherein the anti-DLL3 agent is administered at a dose of from 0.3 mg to 30 mg or from 3 mg to 100 mg once every two weeks, and wherein the anti-PD-1 antibody is administered at a dose approved by the regulatory agency.

“About” or “approximately,” when used in connection with a measurable numerical variable, refers to the indicated value of the variable and to all values of the variable that are within the experimental error of the indicated value (e.g. within the 95% confidence interval for the mean) or ±10% of the indicated value, whichever is greater. Numeric ranges are inclusive of the numbers defining the range.

“first step dose” when used in connection with administration of anti-DLL3 agents for the treatment of cancer (e.g., SCLC) refers to the initial dose of an anti-DLL3 agent in a step dose schedule or regimen. Typically, a first step dose equals to or is lower than a dose at which a first dose effect (e.g., cytokine release syndrome (CRS)) is observed. As known in the art, first step dose can be determined by modeling and simulation of safety and pharmacokinetic data. For example, a first step dose can be a maximum tolerated dose (MTD) of an anti-DLL3 agent where no CRS or a CRS lower than a certain grade (e.g., Grade 2) is observed.

“Target dose” when used in connection with administration of anti-DLL3 agents for the treatment of cancer (e.g., SCLC) refers to a dose that achieves a target effect of an anti-DLL3 agent (e.g., ameliorating or reducing the severity of SCLC, or shortening the length of the SCLC).

“Step dose” when used in connection with administration of anti-DLL3 agents for the treatment of cancer (e.g., SCLC) refers to a dose in a step dose schedule or regimen that is higher than the previous dose at which an anti-DLL3 agent is administered. Step dose includes one or more doses that increase from a first step dose to reach a target dose.

### 2. Agents Targeting DLL3

DLL3 is a non-canonical Notch ligand expressed primarily during embryonic development that functions during somitogenesis. DLL3 accumulate in the Golgi in normal tissues (Geffers et al, J Cell Biol. 178:465-476 (2007)). DLL3 was identified as a tumor-associated antigen and a compelling target for T cell-based therapies by analyzing the differential expression of this target in 28 SCLC tumors and a large panel of normal tissues (Study 123658).

The human DLL3 protein comprises eight extracellular domains: signal peptide, N-terminus, DSL, EGF1, EGF2, EGF3, EGF4, EGF5 and EGF6. The amino acid sequence of human DLL3, the EGF3 domain, the EGF4 domain, and the combined EGF3 and EGF4 domains are shown in the sequence table as SEQ ID NOs: 28, 29, 30 and 31, respectively.

An exemplary agent targeting DLL3 is a bispecific T cell engaging antigen-binding polypeptide that binds DLL3 and CD3, such as a BiTE® molecule. BiTE® molecules are recombinant proteins made from two flexibly linked binding domains, each domain derived from antibodies. One binding domain of BiTE® molecule is specific for a tumor-associated surface antigen (such as DLL3); the second binding domain is specific for CD3, a subunit of the T cell receptor complex on T cells. By their design, BiTE® molecules are uniquely suited to transiently connect T cells with target cells and, at the same time, potently activate the inherent cytolytic potential of T cells against target cells. See e.g., WO 99/54440, WO 2005/040220, and WO 2008/119567.

Accordingly, in some embodiments, the agent targeting DLL3 described comprises two binding domains: the first domain binds DLL3 (preferably human DLL3), and the second domain binds CD3 (preferably human CD3). Preferably, the first domain binds to an epitope of DLL3 comprised within the amino acid sequence of SEQ ID NO: 31. More preferably, the first domain binds to an epitope of DLL3 comprised within the amino acid sequence of SEQ ID NO: 29.

In certain embodiments, the DLL3-binding domain comprises (a) a heavy chain variable region (VH) that comprises: (i) a VH complementarity determining region one (CDR-H1) comprising the amino acid sequence of SEQ ID NO:1; (ii) a CDR-H2 comprising the amino acid sequence of SEQ ID NO:2; and (iii) a CDR-H3 comprising the amino acid sequence of SEQ ID NO:3; and (b) a light chain variable region (VL) that comprises: (i) a VL complementarity determining region one (CDR-L1) comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6.

In certain embodiments, the DLL3-binding domain comprises: a VH that comprises the amino acid sequence of SEQ ID NO:7, and a VL that comprises the amino acid sequence of SEQ ID NO:8. In certain preferred embodiments, the DLL3-binding domain comprises: a VH that comprises the amino acid sequence of SEQ ID NO:11, and a VL that comprises the amino acid sequence of SEQ ID NO:12.

In some embodiments, the VH and VL are joined by a linker to form a single chain Fv (scFv). In some embodiments, the linker is a peptide linker comprising a sequence selected from any one of SEQ ID NOs: 42-50. In some embodiments, the linker is a GS liker, such as Gly-Gly-Gly-Gly-Ser (G4S. SEQ ID NO: 43), or polymers thereof, i.e. (Gly4Ser)x, where x is an integer of 1 or greater (e.g. 2 or 3) (e.g., SEQ ID NOs: 49, 50).

In certain embodiments, the DLL3-binding domain comprises the amino acid sequence of SEQ ID NO: 9. In certain preferred embodiments, the DLL3-binding domain comprises the amino acid sequence of SEQ ID NO: 13.

In certain embodiments, the CD3-binding domain comprises: (a) a VH that comprises: a CDR-H1 comprising the amino acid sequence of SEQ ID NO: 18, a CDR-H2 comprising the amino acid sequence of SEQ ID NO: 19, and a CDR-H3 comprising the amino acid sequence of SEQ ID NO:20; and a VL that comprises: a CDR-L1 comprising the amino acid sequence of SEQ ID NO: 15, a CDR-L2 comprising the amino acid sequence of SEQ ID NO: 16, and a CDR-L3 comprising the amino acid sequence of SEQ ID NO: 17.

In certain embodiments, the CD3-binding domain comprises: a VH that comprises the amino acid sequence of SEQ ID NO:21, and a VL that comprises the amino acid sequence of SEQ ID NO:22. In some embodiments, the VH and VL are joined by a linker to form a single chain Fv (scFv). In some embodiments, the linker is a peptide linker comprising a sequence selected from any one of SEQ ID NOs: 42-50. In some embodiments, the linker is a GS liker, such as Gly-Gly-Gly-Gly-Ser (G4S, SEQ ID NO: 43), or polymers thereof, i.e. (Gly4Ser)x, where x is an integer of 1 or greater (e.g. 2 or 3).

In certain embodiments, the CD3-binding domain comprises the amino acid sequence of SEQ ID NO: 23.

In certain embodiments, the DLL3-binding domain and the CD3-binding domain are joined by a linker. In some embodiments, the linker is a peptide linker comprising a sequence selected from any one of SEQ ID NOs: 42-50. In some embodiments, the linker is a GS liker, such as Gly-Gly-Gly-Gly-Ser (G4S, SEQ ID NO: 43), or polymers thereof, i.e. (Gly4Ser)x, where x is an integer of 1 or greater (e.g., 2 or 3).

In certain embodiments, the anti-DLL3 agent disclosed herein comprises two domains. The first domain binds to DLL3 (preferably human DLL3) and comprises (a) a heavy chain variable region (VH) that comprises: (i) a VH complementarity determining region one (CDR-H1) comprising the amino acid sequence of SEQ ID NO: 1; (ii) a CDR-H2 comprising the amino acid sequence of SEQ ID NO:2; and (iii) a CDR-H3 comprising the amino acid sequence of SEQ ID NO:3; and (b) a light chain variable region (VL) that comprises: (i) a VL complementarity determining region one (CDR-L1) comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6. The second domain binds to CD3 (preferably human CD3), and comprises (a) a VH that comprises: (i) a CDR-H1 comprising the amino acid sequence of SEQ ID NO: 18, (ii) a CDR-H2 comprising the amino acid sequence of SEQ ID NO:19, and (iii) a CDR-H3 comprising the amino acid sequence of SEQ ID NO:20; and (b) a VL that comprises: (i) a CDR-L1 comprising the amino acid sequence of SEQ ID NO:15, (ii) a CDR-L2 comprising the amino acid sequence of SEQ ID NO: 16, and (iii) a CDR-L3 comprising the amino acid sequence of SEQ ID NO: 17.

In certain embodiments, the anti-DLL3 agent described herein comprises two domains: (a) the first domain binds DLL3 (preferably human DLL3) and comprises: a VH that comprises the amino acid sequence of SEQ ID NO:7, and a VL that comprises the amino acid sequence of SEQ ID NO:8; and (b) the second domain binds CD3 (preferably human CD3) and comprises: a VH that comprises the amino acid sequence of SEQ ID NO:21, and a VL that comprises the amino acid sequence of SEQ ID NO:22. In certain preferred embodiments, the anti-DLL3 agent described herein comprises two domains: (a) the first domain binds DLL3 (preferably human DLL3) and comprises: a VH that comprises the amino acid sequence of SEQ ID NO: 11, and a VL that comprises the amino acid sequence of SEQ ID NO: 12; and (b) the second domain binds CD3 (preferably human CD3) and comprises: a VH that comprises the amino acid sequence of SEQ ID NO:21, and a VL that comprises the amino acid sequence of SEQ ID NO:22.

In certain embodiments, the anti-DLL3 agent described herein comprises two domains: (a) the first domain binds DLL3 (preferably human DLL3) and comprises the amino acid sequence of SEQ ID NO: 9, (b) the second domain binds CD3 (preferably human CD3) and comprises the amino acid of SEQ ID NO: 23. In certain embodiments, the anti-DLL3 agent described herein comprises two domains: (a) the first domain binds DLL3 (preferably human DLL3) and comprises the amino acid sequence of SEQ ID NO: 13, (b) the second domain binds CD3 (preferably human CD3) and comprises the amino acid of SEQ ID NO: 23.

In certain embodiments, the anti-DLL3 agent described herein comprises the amino acid sequence of SEQ ID NO: 10. In certain embodiments, the anti-DLL3 agent described herein comprises the amino acid sequence of SEQ ID NO: 14.

In certain embodiments, anti-DLL3 agent described herein further comprises a third domain that extends or enhance the serum half-life of the anti-DLL3 agent. In certain embodiments, the third domain comprises two polypeptides joined by a linker, each peptide comprising a hinge, a CH2 and a CH3 domain of human IgG. In certain embodiments, the third domain comprises, in an N- to C-terminal order: hinge-CH2-CH3-linker-hinge-CH2-CH3. In some embodiments, the linker is a GS liker, such as Gly-Gly-Gly-Gly-Ser (G4S. SEQ ID NO: 43), or polymers thereof, i.e. (Gly4Ser)x, where x is an integer of 1 or greater (e.g., 6). In certain embodiments, the third domain comprises the amino acid sequence selected from any one of SEQ ID NOs: 51-58.

In certain embodiments, the DLL3-binding domain and the CD3-binding domain are joined by a first linker to form a peptide, which is joined to the third domain by a second linker. In certain embodiments, the first linker is a peptide linker comprising a sequence selected from any one of SEQ ID NOs: 42-50, and the second linker comprises a sequence selected from any one of SEQ ID NO: 42, 43, 45, 46, 47, 49 and 50. In some embodiments, the first linker is a GS liker, such as Gly-Gly-Gly-Gly-Ser (G4S, SEQ ID NO: 42), or polymers thereof, i.e. (Gly4Ser)x, where x is an integer of 1 or greater (e.g. 2 or 3), and the second linker comprises a sequence selected from any one of SEQ ID NO: 42, 43, 45, 46, 47, 49 and 50.

In certain embodiments, the anti-DLL3 agent described herein comprises three domains: (a) the first domain binds DLL3 (preferably human DLL3) and comprises the amino acid sequence of SEQ ID NO: 9. (b) the second domain binds CD3 (preferably human CD3) and comprises the amino acid of SEQ ID NO: 23, and (c) the third domain comprises an amino acid sequence selected from any one of SEQ ID NOs: 51-58. In certain embodiments, the anti-DLL3 agent described herein comprises three domains: (a) the first domain binds DLL3 (preferably human DLL3) and comprises the amino acid sequence of SEQ ID NO: 13, (b) the second domain binds CD3 (preferably human CD3) and comprises the amino acid of SEQ ID NO: 23, and (c) the third domain comprises any one of the amino acid sequence selected from SEQ ID NOs: 51-58. In certain embodiments, the anti-DLL3 agent described herein comprises the amino acid sequence of SEQ ID NO: 27. In certain embodiments, the anti-DLL3 agent described herein comprises the amino acid sequence of SEQ ID NO: 59.

The anti-DLL3 agent described herein can be produced by recombinant DNA technology known in the art. For example, the anti-DLL3 agent can be produced by a process wherein a host cell (e.g., Chinese hamster ovary cells) comprising a nucleic acid encoding the anti-DLL3 agent described herein is cultured under conditions allowing the expression of the anti-DLL3 agent and the expressed anti-DLL3 agent is then recovered from the cell culture. In various embodiments, the anti-DLL3 agent is tarlatamab (International Nonproprietary Names for Pharmaceutical Substances (INN): Proposed INN: List 123, WHO Drug Information 34(2): 395-397 (2020)), also known as AMG 757. Tarlatamab is an immunoglobulin scFv-scFv-scFc, anti-[Homo sapiens DLL3 (delta-like ligand 3)] and anti-[Homo sapiens CD3E (CD3 epsilon, Leu-4)], monoclonal antibody single chain (scFv)2-scFc, bispecific; IG single chain scFv-scFv-scFc, anti-DLL3 and anti-CD3E (1-982) [scFv-VH-V-kappa anti-DLL3 (1-241) [VH (Homo sapiens IGHV4-59*01 G49>C (44) (96.9%)-(IGHD)-IGHJ4*01 (100%)) CDR-IMGT [8.7.12] (26-33.51-57.96-107) (1-118)-15-mertris(tetraglycyl-seryl) linker (119-133)-V-KAPPA (Homo sapiens IGKV3-20*01 (91.7%)-IGKJ2*01 Q120>C (234) (90.9%)) CDRIMGT [7.3.9] (160-166.184-186.223-231) (134-241)]-6-merseryl-tetraglycyl-seryl linker (242-247)-scFv-VH-V-lambda antiCD3E (248-496) [VH (Mus musculusIGHV10-1*02 (91.9%)-(IGHD)-IGHJ3*01 (86.7%)/Homo sapiens IGHV3-73*01 (87.0%)-(IGHD)-IGHJ5*01 (100%)) CDR-IMGT [8.10.16] (273-280.298-307.346-361) (248-372)-15-mer-tris(tetraglycyl-seryl) linker (373-387)-V-LAMBDA (Homo sapiens IGLV7-43*01 (85.1%)-IGLJ3*02 (100%)) CDR-IMGT [9.3.9] (413-421.439-441.478-486) (388-496)]-4-mer-tetraglycyl linker (497-500)-scFc (h-CH2-CH3)-(h-CH2-CH3) (501-982) [Homo sapiens IGHG1*03 h-CH2-CH3, nG1 ml (hinge 6-15 (501-510), CH2 R83>C (572), N84.4>G (577), V85>C (582) (511-620), CH3 E12 (636), M14 (638) (621-725), CHS>del) (501-725)-30-merhexakis(tetraglycyl-scryl) linker (726-755)-Homo sapiens IGHG1*03 h-CH2-CH3, nG1 ml (hinge 6-15 (756-765), CH2 R83>C (827), N84.4>G (832), V85>C (837) (766-875), CH3 E12 (891), M14 (893) (876-980), CHS (981-982)) (756-982)]], non-glycosylated, produced in Chinese hamster ovary (CHO) cells; immunomodulator, antincoplastic.

### 3. Agents Targeting PD-1

Programmed Cell Death protein 1 (PD-1), also known as CD279, SLEB2, and hSLE1, is a transmembrane protein expressed on activated T, natural killer (NK) and B lymphocytes, macrophages, dendritic cells (DCs) and monocytes. Notably, PD-1 is highly expressed on tumor-specific T cells (Han et al., Am J Cancer Res 10(3): 727-742 (2020)). PD-1 binds to B7 protein family members, PD-1 Ligand 1 (PD-L1; also referred to as CD279 and B7-H1) and PD-1 Ligand 2 (also known as PD-L2, CD273, and B7-DC). PD-L1 is constitutively expressed on T and B cells, macrophages and dendritic cells, whereas PD-L2 expression is typically restricted to activated DC and macrophages (Xing et al., Oncoimmunology 7(3): c1356144 (2017) (doi: 10.1080/2162402X.2017.1356144)). PD-1 inhibits both adaptive and innate immune responses. The PD-1/PD-L1 axis is involved in the suppression of T cell immune responses in cancer. Antagonists of this pathway have been clinically validated across a number of solid tumor indications. PD-1 inhibitors, nivolumab, pembrolizumab, and cemiplimab, and PD-L1 inhibitors atezolizumab, avelumab, and durvalumab target the PD-1/PD-L1 pathway, and each has been approved by the U.S. Food and Drug Administration (FDA) and/or the European Medicines Agency (EMA) for the treatment of various cancers. Additional exemplary agents targeting PD-1 include tislelizumab, dostarlimab, penpulimab, sintilimab, toripalimab, dostarlimab, camrelizumab, zimberelimab and prolgolimab. In certain embodiments, the PD-1 targeting agent that can be used in the treatment disclosed herein is nivolumab, pembrolizumab, cemiplimab, tislelizumab, dostarlimab, penpulimab, sintilimab, toripalimab, dostarlimab, camrelizumab, zimberelimab or prolgolimab. In certain embodiments, the PD-1 targeting agent is nivolumab, pembrolizumab, cemiplimab, tislelizumab or sintilimab. In certain embodiments, the PD-1 targeting agent is nivolumab or pembrolizumab.

Further additional exemplary agents targeting PD-1 include PD-1 antigen binding proteins (e.g., anti-PD-1 antibodies, antigen binding antibody fragments thereof, and anti-PD-1 antibody protein products) described in International Publication No. WO 2019/140196, which is incorporated herein by reference in its entirety. In exemplary aspects, the PD-1 antigen binding protein binds to human PD-1, which has the amino acid sequence described in National Center for Biotechnology Information (NCBI) Reference Sequence No. NP_005009.2, or SEQ ID NO: 60, or the mature form (e.g., lacking the signal peptide) thereof which is represented by amino acids 21-288 of SEQ ID NO: 60. In exemplary aspects, the PD-1 antigen binding protein binds to cynomolgus PD-1, which has the amino acid sequence described in NCBI Reference Sequence No. NP_001271065.1, or SEQ ID NO: 61, or the mature form thereof. In exemplary instances, the PD-1 antigen binding protein binds to both human PD-1 and cynomolgus PD-1. In exemplary embodiments, the anti-PD-1-antibody comprises the amino acid sequences of SEQ ID NOs: 32-37. In exemplary embodiments, the anti-PD-1-antibody comprises the six CDR amino acid sequences of SEQ ID NOs: 32-37. In exemplary embodiments, the anti-PD-1-antibody comprises a heavy chain (HC) complementarity-determining region (CDR) 1 amino acid sequence of SEQ ID NO: 32, an HC CDR2 amino acid sequence of SEQ ID NO: 33, an HC CDR3 amino acid sequence of SEQ ID NO: 34, a light chain (LC) CDR1 amino acid sequence of SEQ ID NO: 35, an LC CDR2 amino acid sequence of SEQ ID NO: 36, and an LC CDR3 amino acid sequence of SEQ ID NO: 37. In certain embodiments, the anti-PD-1 antibody comprises a PD-1-binding domain comprising (a) a heavy chain variable region (VH) that comprises: (i) a VH complementarity determining region one (CDR-H1) comprising the amino acid sequence of SEQ ID NO:32; (ii) a CDR-H2 comprising the amino acid sequence of SEQ ID NO: 33; and (iii) a CDR-H3 comprising the amino acid sequence of SEQ ID NO:34; and (b) a light chain variable region (VL) that comprises: (i) a VL complementarity determining region one (CDR-L1) comprising the amino acid sequence of SEQ ID NO: 35; (ii) a CDR-L2 comprising the amino acid sequence of SEQ ID NO: 36; and (iii) a CDR-L3 comprising the amino acid sequence of SEQ ID NO: 37. In certain embodiments, the PD-1-binding domain comprises: a VH that comprising the amino acid sequence of SEQ ID NO: 38, and a VL that comprises the amino acid sequence of SEQ ID NO: 39. In certain embodiments, the anti-PD-1-antibody comprises a VH comprising the amino acid sequence of SEQ ID NO: 38 and a VL comprising the amino acid sequence of SEQ ID NO: 39. In certain preferred embodiments, the anti-PD-1-antibody comprises a HC comprising the amino acid sequence of SEQ ID NO: 40 and a LC comprising the amino acid sequence of SEQ ID NO:41. In various instances, the anti-PD-1 antibody is zeluvalimab (International Nonproprietary Names for Pharmaceutical Substances (INN): Proposed INN: List 124, WHO Drug Information 34(4): 929-1102 (2020)), also referred to as AMG 404. Zeluvalimab is an immunoglobulin G1-kappa, anti-[Homo sapiens PDCD1 (programmed cell death 1. PD-1, PD1. CD279)], monoclonal antibody comprising a gamma1 heavy chain (1-450) [VH (Homo sapiens IGHV3-23*03 (92.8%)-(IGHD)-IGHJ3*01 (92.3%)) CDR-IMGT [8.8.13] (26-33.50-58.97-109) (1-120)-Homo sapiens IGHG1*03v. G1m3>G1m17, nG1 ml (CH1 R120>K (217) (121-218), hinge 1-15 (219-233). CH2 R83>C (295). N84.4>G (300). V85>C (305) (234-343). CH3 E12 (359), M14 (361) (344-448). CHS (449-450)) (121-450)]. (223-214′)-disulfide with kappa light chain (1′-214′) [V-KAPPA (Homo sapiens IGKV1-12*01 (96.8%)-IGKJ4*01 (100%)) CDR-IMGT [6.3.9](27-32.50-52.89-97) (1′-107′)-Homo sapiens IGKC*01 (100%). Km3 A45.1 (153), V101 (191) (108′-214′)]; dimer (229-229″:232-232″)-bisdisulfide, produced in Chinese hamster ovary (CHO) cells, non-glycosylated immunomodulator, antineoplastic.

### 3. Dosing Regimen with Agents Targeting DLL3 and PD-1

Disclosed herein are methods of treating DLL3-positive cancer comprising administering to a subject in need thereof with a combination of agents targeting DLL3 and PD-1. Agents targeting DLL3 include anti-DLL3 agents disclosed herein, agents targeting PD-1 include anti-PD-1 antibodies disclosed herein. In one embodiment, disclosed herein is a method of treating DLL3-positive cancer comprising administering to a subject in need thereof with a combination of an ati-DLL3 agent and an anti-PD-1 antibody, wherein the anti-DLL3 agent is administered at a dose of from about 0.3 mg to about 30 mg or from about 3 mg to about 100 mg once every two weeks. In various embodiments, the anti-PD-1 antibody is nivolumab, pembrolizumab, zeluvalimab, or tislelizumab. In certain embodiments, the DLL3-positive cancer is small cell lung cancer (SCLC). In certain embodiments, the SCLC is relapsed/refractory SCLC (RR SCLC) or extensive disease SCLC (ED SCLC). In certain embodiments, the subject is a human having SCLC, e.g., RR SCLC or ED SCLC. In certain embodiments, the SCLC recurred in the subject after at least one prior platinum-based treatment.

In certain embodiments, the anti-DLL3 agent is administered once every two weeks at a dose of: from about 0.3 mg to about 30 mg, from about 1 mg to about 30 mg, from about 3 mg to about 30 mg or from about 10 mg to about 30 mg. In certain embodiments, the anti-DLL3 agent is administered once every two weeks at a dose of about 0.3 mg, 1 mg, 3 mg, 10 mg, 25 mg or 30 mg.

In certain embodiments, the anti-DLL3 agent is administered once every two weeks at a dose of: from about 3 mg to about 100 mg, from about 10 mg to about 100 mg, or from about 30 mg to about 100 mg. In certain embodiments, the anti-DLL3 agent is administered once every two weeks at a dose of about 3 mg, 10 mg. 25 mg, 30 mg, 50 mg, 75 mg or 100 mg.

The anti-DLL3 agent can be administered by any suitable means, including parenteral, intrapulmonary, intranasal, and/or intralesional administration. Parenteral administration includes intramuscular, intravenous, intraarterial, intraperitoneal, or subcutaneous administration. In some embodiments, the anti-DLL3 agent is administered by intravenous (IV) infusion, such as a short IV infusion (approximately 60 minutes), once every two weeks.

In some embodiments wherein the anti-DLL3 agent is administered at a dose described above, the anti-PD-1 antibody is zeluvalimab and the anti-PD-1 antibody is administered at a dose of 480 mg once every four weeks. In certain embodiments, the anti-DLL3 agent and zeluvalimab are administered in a 28-day cycle and both agents can be administered on cycle 1 day 1. To mitigation the possible risk of first dose effect (e.g., cytokine release syndrome (CRS)) of AMG 757 that may be exacerbated with the combination of the anti-PD-1 antibody on cycle 1 day 1, the anti-PD-1 antibody can be administered on cycle 1 day 8 or day 15. Accordingly, in certain embodiments, the anti-DLL3 agent is administered on day 1 and day 15 of a 28-day cycle and the anti-PD-1 antibody is administered on day 1, day 8, or day 15 of a 28-day cycle.

In certain embodiments of such 28-day cycle dosing regimen, the anti-DLL3 agent is administered on day 1 and day 15, zeluvalimab is administered on day 1, day 8 or day 15 in cycle 1, and then on day 1 or day 15 starting in cycle 2 and thereafter. In such embodiments, if zeluvalimab is administered on day 1 or day 8 in cycle 1, then the antibody is administered on day 1 starting in cycle 2 and thereafter; alternatively, if zeluvalimab is administered on day 15 in cycle 1, then the antibody is administered on day 15 starting in cycle 2 and thereafter.

Other known anti-PD-1 antibodies (e.g., pembrolizumab and nivolumab) can also be used in combination with the anti-DLL3 agent in the methods disclosed herein. When used in the combination, the dose and regimen of these other anti-PD-1 antibodies are the same as approved by regulatory agencies (e.g., the FDA). For example, as described in Example 1, the anti-DLL3 agent was used in combination with pembrolizumab in a clinical study in patients with SCLC wherein pembrolizumab was administered at a dose of 200 mg every three weeks. Thus, in some embodiments wherein the anti-DLL3 agent is administered at a dose described above, the anti-PD-1 antibody is pembrolizumab and the anti-PD-1 antibody is administered at a dose of 200 mg once every three weeks. In some embodiments wherein the anti-DLL3 agent is administered at a dose described above, the anti-PD-1 antibody is nivolumab and the anti-PD-1 antibody is administered at a dose of 240 mg once every two weeks. In some embodiments wherein the anti-DLL3 agent is administered at a dose described above, the anti-PD-1 antibody is tislelizumab and the anti-PD-1 antibody is administered at a dose of 200 mg once every three weeks.

In embodiments wherein the anti-DLL3 agent is administered once every two weeks and the anti-PD-1 antibody is administered once every three weeks, the anti-PD-1 antibody can start on day 15 in cycle 1 to minimize possible risk of first dose effect (e.g., CRS). Accordingly, in certain embodiments, the first cycle wherein the anti-DLL3 agent and the anti-PD-1 antibody are administered is a 28 day cycle, the anti-DLL3 agent is administered on day 1 and day 15 and the anti-PD-1 antibody is administered on day 15, thereafter, the anti-DLL3 agent is administered once every two weeks and the anti-PD-1 antibody is administered once every three week.

The anti-PD-1 antibody can be administered by any suitable means, including parenteral. In some embodiments, the anti-PD-1 antibody is administered by intravenous IV infusion, once every two weeks, once every three weeks, or once every four weeks depending on the antibody.

As used herein, “combination therapy” or “in combination with” refers to administration of one treatment modality (e.g., an anti-DLL3 agent) in addition to another treatment modality (e.g., an anti-PD-1 antibody). As such, “combination therapy” or “in combination with” refers to administration of one treatment modality before, during, or after administration of the other treatment modality to an individual (e.g., a human having SCLC). However, combination therapy does not include situations wherein 28 or more days have elapsed between the end of administration of one treatment modality and the beginning of another treatment modality.

### 3.1 Step Dosing

Due to its mechanism of action, subjects may be at an increased risk for first dose effects (e.g., CRS) following initial infusion of AMG 757, which may be exacerbated with the combination of anti-PD-1 antibody. To mitigate the risk, step dosing regimens can be implemented. For example, if a first dose effect (e.g., CRS) is experienced by a subject, an appropriate first dose not exceeding the dose at which a CRS event is observed can be determined and implemented. One or more step doses can also be determined and implemented until a target dose is reached. These doses and dosing schedules can be guided by emerging pharmacokinetics and safety data and information available for AMG 757.

Exemplary step dosing schedules of anti-DLL3 agents (e.g., AMG 757) in a 28-day cycle are shown in the table below (cycle 1 only), the anti-DLL3 agent is administered once every two weeks thereafter.

TABLE 1
Exemplary Single and Multiple Step Dosing Schedules (Cycle 1 only)

| Anti-DLL3 agent | | | | |
|---|---|---|---|---|
| (AMG 757) | Day 1 | Day 4 | Day 8 | Day 15* |
| One-step | First | N/A | Step dose | Target dose |
| | step dose | | (equal to target dose) | |
| Two-step | First | Step | Step dose | Target dose |
| (Option 1) | step dose | dose** | (equal to target dose) | |
| Two-step | First | N/A | Step dose | Step dose |
| (Option 2) | step dose | | | (equal to target dose) |
| Three-step | First | Step | Step dose** | Step dose |
| | step dose | dose | | (equal to target dose) |

*AMG 757 administered at the same dose as day 15 every 2 weeks thereafter.
**Based on emerging pharmacokinetic, pharmacodynamic, and safety data in the current study as well as the ongoing FIH study (20160323), the step dose on day 4 in two-step dosing (option 1) or the step dose on day 8 in three-step dosing may be equal to the target dose.

Accordingly, disclosed herein are methods of treating DLL3-positive cancer comprising administering to a subject in need thereof an anti-DLL3 agent and an anti-PD-1 antibody, wherein the anti-DLL3 agent is administered according to a step dosing schedule such as those outlined in Table 1 above. The anti-PD-1 antibody can be nivolumab, pembrolizumab, zeluvalimab, or tislelizumab. For example, the anti-PD-1 antibody is zeluvalimab and is administered at a dose of 480 mg once every four weeks in various embodiments wherein a step dosing regimen is implemented for AMG 757.

In certain embodiments, the anti-DLL3 agent is administered according to a one-step dosing schedule. In such embodiments, disclosed herein is a method of treating DLL3-positive cancer comprising administering to a subject in need thereof an anti-DLL3 agent and an anti-PD-1 antibody, wherein the anti-DLL3 agent is administered in a 28-day cycle according to the following schedule: a) a first dose of about 0.3 mg or 1 mg on cycle 1 day 1, b) a second dose on cycle 1 day 8, c) a third dose on cycle 1 day 15, and d) one or more subsequence doses starting on cycle 2 day 1 and once every two weeks thereafter, and wherein the second, the third and the subsequent doses are the same, are each from about 0.3 mg to about 30 mg or from about 3 mg to about 100 mg, and are higher than the first dose. In some embodiments, the anti-PD-1 antibody is zeluvalimab and is administered at a dose of about 480 mg once every four weeks. In some embodiments, the anti-PD-1 antibody is nivolumab, pembrolizumab, or tislelizumab administered at a dose/regimen approved by a regulatory agency.

In certain embodiments wherein the anti-DLL3 agent is administered according to the one-step dosing schedule, the first dose of the anti-DLL3 agent is about 0.3 mg or 1 mg, the second, third and subsequent doses are each of: from about 0.3 mg to about 30 mg, from about 1 mg to about 30 mg, from about 3 mg to about 30 mg or from about 10 mg to about 30 mg. In certain embodiments, the first dose of the anti-DLL3 agent is about 0.3 mg, the second, third and subsequent doses are each of about 1 mg. 3 mg, 10 mg, 25 mg or 30 mg. In certain embodiments, the first dose of the anti-DLL3 agent is about 1 mg, the second, third and subsequent doses are each of about 3 mg, 10 mg. 25 mg or 30 mg.

In certain embodiments wherein the anti-DLL3 agent is administered according to the one-step dosing schedule, the first dose of the anti-DLL3 agent is about 0.3 mg or 1 mg, the second, third and subsequent doses are each of from about 3 mg to about 100 mg, from about 10 mg to about 100 mg, or from about 30 mg to about 100 mg. In certain embodiments, the first dose is about 0.3 mg, the second, third, and subsequent doses are each of about 1 mg, 10 mg, 25 mg, 30 mg, 50 mg, 75 mg, or 100 mg. In certain embodiments, the first dose is about 1 mg, the second, third, and subsequent doses are each of about 10 mg. 25 mg, 30 mg, 50 mg, 75 mg, or 100 mg.

In certain embodiments, the anti-PD-1 antibody is zeluvalimab and is administered on day 1, day 8 or day 15 in cycle 1, and then on day 1 or day 15 starting in cycle 2 and thereafter. If the anti-PD-1 antibody is administered on day 1 or day 8 in cycle 1, then the antibody is administered on day 1 starting in cycle 2 and thereafter. Alternatively, if zeluvalimab is administered on day 15 in cycle 1, then the antibody is administered on day 15 in cycle 2 and thereafter.

In certain embodiments, the anti-DLL3 agent is administered according to a two-step dosing schedule. Thus, disclosed herein are methods of treating DLL3-positive cancer comprising administering to a subject in need thereof an anti-DLL3 agent and an anti-PD-1 antibody, wherein the anti-DLL3 agent is administered in a 28-day cycle according to Schedule I or Schedule II below, the anti-PD-1 antibody is administered at a dose of 480 mg once every four weeks, and wherein
Schedule I: a) a first dose (first step dose) of 0.3 mg or 1 mg on cycle 1 day 1, b) a second dose (step dose) on cycle 1 day 4, c) a third dose (step dose, equal to target dose) on cycle 1 day 8, d) a fourth dose (target dose) on cycle 1 day 15, and e) one or more subsequence doses (target dose) starting on cycle 2 day 1 and once every two weeks thereafter, and wherein the second dose is higher than the first dose, and the third, the fourth and the subsequent doses are the same, are each from about 0.3 mg to 30 mg or from 3 mg to 100 mg, and are higher than the second dose; or
Schedule II: a) a first dose (first step dose) of 0.3 mg or 1 mg on cycle 1 day 1, b) a second dose (step dose) on cycle 1 day 8, c) a third dose (step dose, equal to target dose) of on cycle 1 day 15 and c) one or more subsequence doses (target dose), starting on cycle 2 day 1 and once every two weeks thereafter, and wherein the second dose is higher than the first dose, and the third dose and subsequent doses are the same, are each from about 0.3 mg to 30 mg or from 3 mg to 100 mg, and are higher than the second dose.

In certain embodiments, if pharmacokinetic and safety data are deemed to be satisfactory, the step dose on cycle 1 day 4 of Schedule I described above can be higher than or equal to the target dose. However, no step dose or target dose exceeds the amount of 100 mg. It is believed that such dosing schedules are beneficial in that they may lead to improved PD activity (e.g., help to achieve the desired serum AMG 757 levels quickly).

In certain embodiments, disclosed herein are methods of treating DLL3-positive cancer comprising administering to a subject in need thereof an anti-DLL3 agent, wherein the anti-DLL3 agent is administered according to a three-step dosing schedule. In such embodiments, disclosed herein are methods of treating DLL3-positive cancer comprising administering to a subject in need thereof an anti-DLL3 agent and an anti-PD-1 antibody, wherein said anti-DLL3 agent is administered in a 28-day cycle according to the following schedule: a) a first dose (first step dose) of about 0.3 mg or 1 mg on cycle 1 day 1, b) a second dose (step dose) on cycle 1 day 4, c) a third dose (step dose) on cycle 1 day 8, d) a fourth dose (step dose, equal to target dose) on cycle 1 day 15, and e) one or more subsequence doses (target dose) starting on cycle 2 day 1 and once every two weeks thereafter, and wherein the second dose is higher than the first dose, the third dose is higher than the second dose, and the fourth dose and the subsequent doses are the same, are each from about 0.3 mg to about 30 mg or from about 3 mg to about 100 mg, and are higher than the third dose, and wherein the anti-PD-1 antibody is nivolumab, pembrolizumab, zeluvalimab, or tislelizumab administered at a dose and schedule described above. In certain embodiments, the anti-PD-1 antibody is zeluvalimab and is administered at a dose of about 480 mg once every four weeks. In other embodiments, the anti-PD-1 antibody is pembrolizumab and is administered at a dose of 200 mg once every three weeks.

In certain embodiments, if pharmacokinetic and safety data are deemed to be satisfactory, the step dose on cycle 1 day 8 of the three-step dosing regimen described above can be equal to the target dose. It is believed that such dosing schedules are beneficial in that they help to achieve the desired serum AMG 757 levels quickly.

The anti-DLL3 agent and the anti-PD-1 antibody can be administered by any suitable means, including parenteral, intrapulmonary, intranasal, and/or intralesional administration. Parenteral administration includes intramuscular, intravenous, intraarterial, intraperitoneal, or subcutaneous administration. In some embodiments, the anti-DLL3 agent is administered by IV infusion, and the anti-PD-1 antibody is administered by IV infusion.

In certain embodiments, the DLL3-positive cancer is small cell lung cancer (SCLC). In certain embodiments, the SCLC is relapsed/refractory SCLC (RR SCLC) or extensive disease SCLC (ED SCLC). In certain embodiments, the subject is a human having SCLC, e.g., RR SCLC or ED SCLC, in certain embodiments, the SCLC recurred in the subject after at least one prior platinum-based chemotherapy.

### 3.2 Additional Therapeutic Agents

In some embodiments, the methods disclosed herein further comprises the use of one or more additional therapeutic agents to prevent, reduce or mitigate the risk of adverse effects associated with the administration of the anti-DLL3 agent and the anti-PD-1 antibody. A major adverse effect associated with the use of the anti-DLL3 agent is CRS. The one or more additional therapeutic agents useful for prevent, reduce or mitigate the risk of CRS include corticosteroids (e.g., dexamethasone), fluid (e.g., saline), etanercept (e.g., Enbrel) and anti-IL6 antibody (e.g., tocilizumab or siltuximab). Dexamethasone may be administered by IV administration prior to all cycle 1 doses of AMG 757 including all step doses, saline (e.g., 1 liter) may be administered IV following all AMG 757 doses in cycle 1, and anti-IL6 antibody (tocilizumab or siltuximab) may be administered as needed (e.g., subject not responsive to IV fluid). Exemplar dose of dexamethasone includes 8 mg/administration (maximum of 24 mg/day). Exemplary dose of tocilizumab includes 8 mg/kg (not to exceed 800 mg). Symptoms of CRS include fever, nausea, fatigue, headache, myalgias, malaise, and therapeutic agents useful for treating such these symptoms (e.g., paracetamol/acetaminophen for fever) may also be used.

Adverse events following the administration of the anti-PD-1 antibody may include immune-related adverse reactions that may occur shortly after the first dose to several months after the last dose of treatment. Immune-related adverse reactions associated with the anti-PD-1 antibody include pneumonitis, colitis/diarrhea, immune-mediated hepatitis, adrenal insufficiency, nephritis and renal dysfunction, encephalopathy, rash on the skin, hypothyroidism, hyperthyroidism, and diabetes mellitus. One or more additional therapeutic agents useful for prevent, reduce or mitigate the risk of such immune-related adverse reactions (e.g., one or more of pneumonitis, colitis/diarrhea, immune-mediated hepatitis, adrenal insufficiency, nephritis and renal dysfunction, encephalopathy, rash on the skin, hypothyroidism, hyperthyroidism, and diabetes mellitus) include corticosteroids (e.g., prednisone, hydrocortisone, and dexamethasone), insulin therapy (for diabetes mellitus), thyroid hormone supplementation (for hypothyroidism), and B-Blocker (e.g., atenolol, propranolol for hyperthyroidism).

Thus, in certain embodiments, the methods disclosed herein further comprise administering one or more additional therapeutic agents selected from a corticosteroid (e.g., prednisone, hydrocortisone, and dexamethasone), a fluid (saline), anti-IL-6 antibody (e.g., tocilizumab or siltuximab), insulin therapy, thyroid hormone supplementation, and a β-Blocker (e.g., atenolol, propranolol). In certain embodiments, the methods further comprise administering one or more additional therapeutic agents selected from a corticosteroid (e.g., dexamethasone), a fluid (saline) and tocilizumab or siltuximab. In certain embodiments, the one or more of the corticosteroid, fluid and anti-IL-6 antibody (e.g., tocilizumab or siltuximab) are administered in cycle 1 wherein AMG 757 is administered.

In certain embodiments of any one of methods wherein one or more additional therapeutic agents are administered, the subject is a human.

### 4. Articles of Manufacture

Disclosed herein are articles of manufacture comprising: (a) a container comprising an anti-DLL3 agent; and (b) a package insert with instructions for treating DLL3-positive cancer (or treating SCLC) in a subject by administering the anti-DLL3 agent (e.g., AMG 757) in combination with an anti-PD-1 antibody (e.g., pembrolizumab or AMG 404), wherein the instructions specifies that the anti-DLL3 agent is administered at a dose of from about 0.3 mg to about 30 mg or from about 3 mg to about 100 mg (or any of the dose ranges disclosed herein) to the subject once every two weeks, such as on day 1 and day 15 of a 28-day cycle. In certain embodiments, the article of manufacture further comprises a container comprising the anti-PD-1 antibody.

The instruction may also specify that the anti-DLL3 agent be administered in a 28-day cycle according to the following schedule: a) a first dose of 0.3 mg or 1 mg on cycle 1 day 1, b) a second dose on cycle 1 day 8, c) a third dose on cycle 1 day 15, and d) one or more subsequence doses starting on cycle 2 day 1 and once every two weeks thereafter, the second, third, and subsequent doses are the same, are each from 0.3 mg to 30 mg or from 3 mg to 100 mg (or any of the dose ranges disclosed herein), and are higher than the first dose.

The instructions may also specify that the anti-PD-1 antibody be administered on day 1, day 8 or day 15 of the 28-day cycle, for example, the anti-PD-1 antibody be administered on day 1, day 8 or day 15 in cycle 1, then on day 1 or day 15 starting in cycle 2 and thereafter. The instructions may also specify that if the anti-PD-1 antibody is administered on day 1 or day 8 in cycle 1, then it be administered on day 1 starting in cycle and thereafter; alternatively, if the anti-PD-1 antibody is administered on day 15 in cycle 1, then it be administered on day 15 starting in cycle and thereafter.

The instructions may further specify that one or more therapeutic agents be administered to the subject in addition to the anti-DLL3 agent and the anti-PD-1 antibody. The one or more therapeutic agents can be selected from corticosteroid (e.g., such as dexamethasone, prednisone, hydrocortisone), saline, etanercept and anti-IL6 antibody (tocilizumab or siltuximab). In certain embodiments, the instruction specifies that one or more of dexamethasone, saline and anti-IL6 antibody (tocilizumab or siltuximab) be administered in the first cycle in which the anti-DLL3 agent is administered. In certain embodiments, the instruction specifies that dexamethasone is further administered in the first cycle in which the anti-DLL3 agent is administered (e.g., by IV administration prior to cycle 1 doses of the anti-DLL3 agent).

### 5. Subjects

In various instances of the presently disclosed methods, the subject is a human subject. In exemplary instances, the human subject has Small Cell Lung Cancer (SCLC), optionally, a histologically or cytologically confirmed SCLC. In various aspects, the human is male or female and/or greater than or equal to 18 years of age with a SCLC. In exemplary aspects, the human subject has been treated with a platinum-based chemotherapy. In exemplary aspects, the human subject has RR SCLC, optionally, which progressed or recurred following at least one platinum-based chemotherapy. In exemplary instances, the human subject has an Eastern Cooperative Oncology Group (ECOG) performance status of 0-1 (Oken et al., Am J Clin Oncol 5: 649-655 (1982). In various aspects, the human subject has one or more brain metastases that have been treated. In various aspects, the platinum-based chemotherapy comprises carboplatin or cisplatin, platinum-etoposide or platinum-irinotecan.

### 6. Cancer

In various aspects, the cancer treated by the presently disclosed methods is a DLL3-positive cancer. In various instances, the cancer treated by the presently disclosed methods is a small cell lung cancer (SCLC). In exemplary aspects, the SCLC is a histologically or cytologically confirmed SCLC. Optionally, the SCLC is measurable by modified Response Criteria in Solid Tumors (RECIST) 1.1, wherein measurable lesions include (a) non-nodal lesions with clear borders that can be measured accurately and serially in one dimension in the axial plane (longest diameter ≥10 mm measured by magnetic resonance imaging/computed tomography (MRI/CT) with scan slice thickness ≤5 mm) and/or (b) nodal lesions with the longest diameter perpendicular to the long axis (short axis) ≥15 mm on MRI/CT, and/or exclude simple cysts, pleural/pericardial effusions and ascites.

### EXAMPLES

### Example 1 Safety, Tolerability, PK, and Anti-Tumor Activity of AMG 757 in Combination with Pembrolizumab in Subjects with SCLC

A clinical study was carried out using AMG 757 in combination with pembrolizumab in subjects with SCLC. The primary objectives for the study are to evaluate the safety and tolerability of AMG 757 when administered in combination with pembrolizumab and to determine maximum tolerated dose (MTD) or recommended phase 2 dose (RP2D) of AMG 757 in combination with pembrolizumab. The secondary objectives for the study are to characterize the PK of AMG 757 when administered in combination with pembrolizumab and to evaluate preliminary anti-tumor activity of AMG 757 in combination with pembrolizumab.

Key eligibility criteria are summarized in the table below

| Key inclusion criteria | Key exclusion criteria |
|---|---|
| Male or female ≥18 years of age with | History of other malignancy within the past 2 |
| Histologically or cytologically confirmed Small | years prior to first dose of AMG 757 with |
| Cell Lung Cancer (SCLC) | exceptions |
| RR SCLC who progressed or recurred following | Major surgery within 28 days of first dose AMG |
| at least one platinum-based chemotherapy | 757 |
| Eastern Cooperative Oncology Group (ECOG) | Prior anti-cancer therapy: at least 28 days must |
| performance status of 0-2 | have elapsed between any prior anti-cancer |
| | therapy and first dose of AMG 757 |
| Subjects with treated brain metastases are eligible | |
| provided they meet defined criteria | |
| Adequate organ function as defined in protocol | |

The starting dose of AMG 757 was 0.1 mg IV once every two weeks. The dose of AMG 757 was to be escalated as follows: 0.1 mg, 0.3 mg, 1 mg, 3 mg, 10 mg, 30 mg, and 100 mg via IV once every two weeks. The dose of pembrolizumab was fixed at 200 mg IV every 3 weeks. First dose of pembrolizumab was administered on cycle 1 day 15.

As of April 2022, 8 subjects were treated with the combination of AMG 757 and pembrolizumab. The subjects were dosed with pembrolizumab 200 mg IV every 3 weeks and either 0.1 mg (N=5) or 0.3 mg (N=3) of AMG 757 IV every 2 weeks. Among the 8 subjects, 3 subjects achieved stable disease as best overall response delivering an objective response rate of 0% and a disease control rate of 37.5%. One study subject continued on treatment 22 months after first dose of AMG 757 in June 2020 with a response of stable disease.

All subjects experienced at least one treatment emergent adverse event with the most common being fatigue in ⅝ (62.5%). One subject (0.3 mg AMG 757) experienced a treatment related adverse event of interest (Grade ≥3 CRS). No subject had treatment-emergent adverse event(s) that led to treatment discontinuation. No fatal adverse events were recorded for the combination at the doses explored in the subjects.

### Example 3 Study Evaluating the Safety and Efficacy of AMG 757 in Combination with AMG 404 in Subjects with SCLC

### Objectives and Endpoints

The objectives and endpoints of this study (Study 20200439) is summarized in the table below.

| | |
|---|---|
| Objectives | Endpoints |

Primary

| | |
|---|---|
| To evaluate the safety, tolerability, and | Dose-limiting toxicities (DLTs), treatment- |
| recommended phase 2 target dose of AMG 757 | emergent and treatment-related adverse events, |
| in combination with AMG 404 | changes in vital signs, electrocardiograms |
| | (ECGs), and clinical laboratory tests |

Secondary

| | |
|---|---|
| To evaluate anti-tumor activity of AMG 757 in | Objective response (OR) per modified response |
| combination with AMG 404 | evaluation criteria in solid tumors (RECIST) |
| | v1.1, duration of response (DOR), disease control |
| | rate (DCR), progression-free survival (PFS), and |
| | overall survival (OS). |
| To characterize the pharmacokinetics (PK) of | PK parameters including, but not limited to, |
| AMG 757 in combination with AMG 404 | maximum serum concentration (Cmax), |
| | minimum serum concentration (Cmin), and area |
| | under the concentration-time curve (AUC) over |
| | the dosing interval |

Exploratory

| | |
|---|---|
| To evaluate protein, nucleic acid, and cellular | Changes in protein, nucleic acid, and cellular |
| biomarkers in blood pre- and post-treatment | biomarkers in blood |
| Evaluate relationship of baseline target protein | Cell surface protein expression (e.g., DLL3, PD- |
| expression in tumor tissue, tumor | L1) and tumor infiltrating lymphocyte status in |
| microenvironment characteristics, and clinical | tumor tissue at baseline |
| benefit | |
| To evaluate the immunogenicity of AMG 757 | Incidence of anti-AMG 757 antibody and anti- |
| and AMG 404 | AMG 404 antibody formation |

### Study Design

Study 20200439 is a phase 1b, multicenter, open-label study evaluating the safety, tolerability, PK, PD, and efficacy of AMG 757 in combination with AMG 404 in subjects with SCLC. The study consists of dose exploration (Part 1) and dose expansion (Part 2).

The dose exploration part of the study estimates the recommended phase 2 target dose of AMG 757 in combination with AMG 404 using a modified toxicity probability interval (mTPI-2) design. A combination RP2D may be identified based on emerging safety, efficacy, and pharmacodynamic data prior to reaching an MTD.

AMG 404 is administered as a short-term IV infusion (30 minutes) at the dose of 480 mg every 28 days (+3 days) throughout the study. The starting dose of AMG 757 is 1 dose level below the recommended phase 2 target dose as determined in the ongoing FIH study (Study 20160323). Planned dose levels in Study 20160323 are 0.003 mg. 0.01 mg, 0.03 mg, 0.1 mg, 0.3 mg, 1 mg, 3 mg, 10 mg, 30 mg and 100 mg. The highest planned target dose of AMG 757 does not exceed 100 mg in this combination study.

To mitigate the risk of CRS and to potentially optimize the PD activity of AMG 757, a step dosing approach is implemented as part of the initial dosing schedule. Based on the recommended phase 2 target dose and the associated dosing schedule selected in Study 20160323, one of the following step dosing schedules is implemented: one-step, two-step (option 1 or option 2), or three-step. AMG 404 is administered at the dose of 480 mg beginning on cycle 1 day 1. Based on emerging safety data, the dosing schedule may be adjusted to allow for AMG 404 to be administered initially on cycle 1 day 8 or cycle 1 day 15. Depending on which day AMG 404 is administered on in cycle 1, beginning in cycle 2. AMG 404 is administered every 4 weeks beginning on cycle 2 day 1 or cycle 2 day 15 (note if AMG 404 is initially administered on cycle 1 day 8 there is a 21-day interval between the cycle 1 day 8 and cycle 2 day 1 dose).

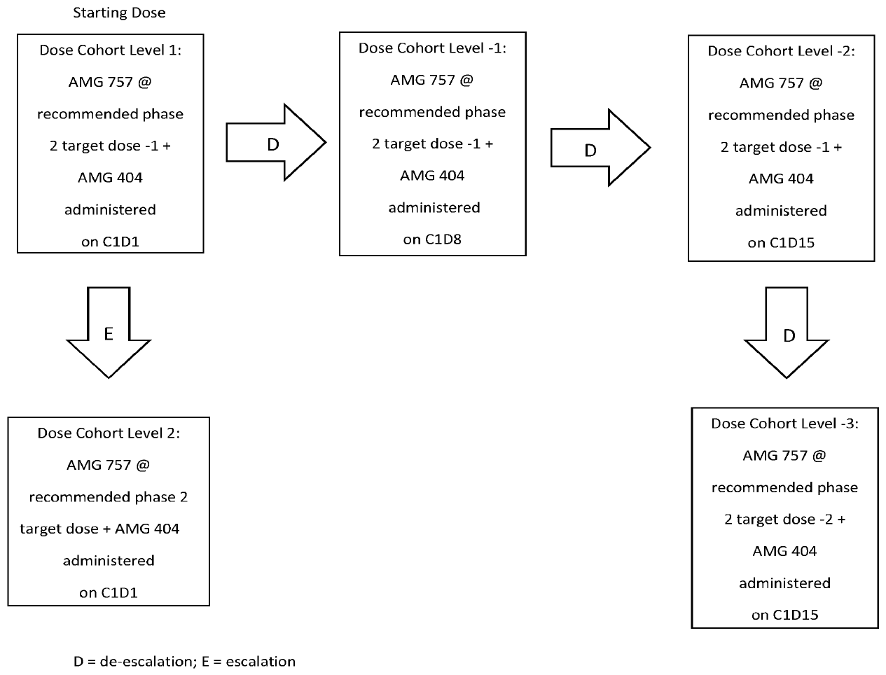

Part 1 may include one or more of the following planned dose levels of AMG 757 in combination with a fixed dose of AMG 404 (see FIG. 1):
Dose Cohort Level 1: AMG 757 at 1 dose level below recommended phase 2 target dose administered IV Q2W (with step dosing) in combination with AMG 404 at 480 mg IV every 4 weeks (Q4W) beginning on cycle 1 day 1
Dose Cohort Level 2: AMG 757 at the recommended phase 2 target dose administered IV Q2W (with step dosing) in combination with AMG 404 at 480 mg IV Q4W beginning on cycle 1 day 1
Dose Cohort Level-1: AMG 757 at 1 dose level below the recommended phase 2 target dose administered IV Q2W (with step dosing) in combination with AMG 404 at 480 mg IV Q4W (in case Dose Cohort Level 1 is not well tolerated) beginning on cycle 1 day 8
Dose Cohort Level-2: AMG 757 at 1 dose level below the recommended phase 2 target dose administered IV Q2W (with step dosing) in combination with AMG 404 at 480 mg IV Q4W (in case Dose Cohort Level-1 is not well tolerated) beginning on cycle 1 day 15
Dose Cohort Level-3: AMG 757 at 2 dose levels below the recommended phase 2 target dose administered IV Q2W (with step dosing) in combination with AMG 404 at 480 mg IV Q4W (in case Dose Cohort Level-2 is not well tolerated) beginning on cycle 1 day 15

Based upon emerging PK, PD, and safety data, alternative (intermediate) dose cohort levels, including adjusting the dose of AMG 757 prior to adjusting the day of AMG 404 administration in cycle 1 as part of the de-escalation recommendations per the DLRM, or alternative dosing schedule(s), including additional step dosing strategies of AMG 757, may be explored.

Dose escalation/de-escalation recommendations is guided by a mTPI-2 model (Guo et al, 2017) with a target toxicity probability of 30%, equivalence toxicity interval of (25%, 33%) and probability of overdosing of 95%. Beta (1, 1) is used as a prior distribution.

Step Dosing: subjects may have an increased risk for cytokine release syndrome during initiation of AMG 757 treatment. It is believed that an optimal recommended phase 2 target dose may require modifications to the step dosing approach. Additionally, to optimize the PD activity of AMG 757 and AMG 404, modifications to the step dosing approach may be required.

Step dosing schedules are summarized below. The dosing schedule may be adapted to include 1 or more of the following measures, as per DLRT recommendation based on emerging safety and PD data:
One-step dosing involving a first step dose on day 1, followed by a step dose on day 8 (equal to the target dose) and the target dose on day 15 then Q2W.
Two-step dosing (option 1) involving a first step dose on day 1, followed by a step dose on day 4, a step dose on day 8 (equal to target dose) and the target dose on day 15 then Q2W.
Two-step dosing (option 2) involving a first step dose on day 1, followed by a step dose on day 8, a step dose on day 15 (equal to target dose) and the target dose on C2D1 then Q2W.
Three-step dosing involving a first step dose on day 1, followed by a step dose on day 4, a step dose on day 8, a step dose on day 15 (equal to target dose) and the target dose on C2D1 then Q2W.

If PK, PD and safety data are deemed to be satisfactory, the step dose on cycle 1 day 4 of Option 1 described above can be higher than or equal to the target dose. However, no step dose or target dose exceeds the amount of 100 mg.

Part 2 (Dose Expansion): Upon completion of Part 1 of the study, enrollment commences in Part 2 to confirm the safety and tolerability of the selected dose and to further evaluate the efficacy of AMG 757 in combination with AMG 404.

Table 2 summaries the Eligibility Criteria for 20200439

TABLE 2
Key Eligibility Criteria

| Key inclusion criteria | Key exclusion criteria |
|---|---|
| Male or female ≥18 years of age with | Other medical conditions: including History of |
| Histologically or cytologically confirmed SCLC | other malignancy within the past 2 years with |
| who progressed or recurred following at least 1 | exceptions, Other medical conditions: including |
| platinum-based regimen | History of other malignancy within the past 2 |
| | years with exceptions, Major surgery within 28 |
| | days of first dose of AMG 757, Untreated or |
| | symptomatic brain metastases and |
| | leptomeningeal disease, Participants who |
| | experienced recurrent grade 2 pneumonitis or |
| | severe or life-threatening immunemediated |
| | adverse events or infusion-related reactions |
| | including those that lead to permanent |
| | discontinuation while on treatment with immuno- |
| | oncology agents, History of any immune-related |
| | colitis. Infectious colitis is allowed if evidence of |
| | adequate treatment and clinical recovery exists |
| | and at least 3 months interval observed since |
| | diagnosis of colitis, Participants with evidence of |
| | interstitial lung disease or active, non-infectious |
| | pneumonitis, Has a diagnosis of |
| | immunodeficiency or is receiving systemic |
| | steroid therapy or any other form of |
| | immunosuppressive therapy within 7 days prior |
| | to the first dose of AMG 757, History of solid |
| | organ transplantation, History of hypophysitis or |
| | pituitary dysfunction, Active autoimmune disease |
| | that has required systemic treatment (except |
| | replacement therapy) within the past 2 years or |
| | any other diseases requiring immunosuppressive |
| | therapy while on study. Participants with Type I |
| | diabetes, vitiligo, psoriasis, hypo- or |
| | hyperthyroid disease not requiring |
| | immunosuppressive treatment are permitted |
| Subjects with disease measurable by modified | |
| Response Evaluation Criteria in Solid Tumors | |
| (RECIST) 1.1 | |
| Eastern Cooperative Oncology Group (ECOG) | Prior/Concomitant therapy as defined in the |
| performance status of 0-1 | protocol: including anti-PD1 or antiPDL1 |
| | antibody therapy, at least 28 days must have |
| | elapsed between any prior anti-cancer therapy |
| | and first dose of AMG 757 |
| | Exception: Participants who received prior |
| | chemotherapy must have completed at least 14 |
| | days before the first dose of AMG 757 and all |
| | treatment-related toxicity resolved to grade ≤1. |
| | Participants who received prior palliative |
| | radiotherapy must have completed at least 7 days |
| | before the first dose of AMG 757 |
| Subjects with treated brain metastases are eligible | Prior/concurrent clinical study experience as |
| provided they meet defined criteria | defined in protocol |
| Adequate organ function as defined in protocol | |

As of April 2022, 5 subjects were treated with AMG 757 10 mg Q2W with 1 step dosing and AMG 404 480 mg Q4W in this study. Two subjects had unconfirmed partial response, one completed 6 cycles of treatment and the other completed 2 cycles of treatment. The remaining subjects were in cycle 1 of the treatment. No subject had treatment-emergent adverse events greater than grade 2. One subject had a grade 5 event that was due to underlying disease and not related to the treatment. No subject had treatment-emergent adverse event(s) that led to treatment discontinuation.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

1. A method of treating a subject with small cell lung cancer (SCLC) comprising administering to the subject in need thereof an anti-DLL3 agent comprising the amino acid sequence of SEQ ID NOs: 13 and 23 and an anti-PD-1 antibody, wherein the anti-DLL3 agent is administered at a dose of from 0.3 mg to 30 mg or from 3 mg to 100 mg once every two weeks, and wherein the SCLC recurred in the subject after at least one prior platinum-based chemotherapy.

2. The method of claim 1, wherein the anti-DLL3 agent is administered at a dose of from 1 mg to 30 mg once every two weeks.

3. The method of claim 1, wherein the anti-DLL3 agent is administered at a dose of 3 mg, 10 mg, 25 mg or 30 mg once every two weeks.

4. The method of claim 1, wherein the anti-DLL3 agent is administered at a dose of from 10 mg to 100 mg once every two weeks.

5. The method of claim 1, wherein the anti-DLL3 agent is administered at a dose of 10 mg, 25 mg, 30 mg, 50 mg, 75 mg or 100 mg once every two weeks.

6. The method of claim 1, wherein the anti-DLL3 agent is administered on day 1 and day 15 of a 28-day cycle.

7. The method of claim 6, wherein the anti-PD-1 antibody is nivolumab, pembrolizumab, or zeluvalimab.

8. The method of claim 7, wherein the anti-PD-1 antibody is pembrolizumab and wherein the pembrolizumab is administered at a dose of 200 mg once every three weeks.

9. The method of claim 7, wherein the anti-PD-1 antibody is nivolumab and wherein the nivolumab is administered at a dose of 240 mg once every two weeks.

10. The method of claim 7, wherein the anti-PD-1 antibody is zeluvalimab and wherein the zeluvalimab is administered at a dose of 480 mg once every three weeks.

11. The method of claim 8, wherein pembrolizumab is administered before the anti-DLL3 agent if both are administered on the same day.

12. The method of claim 1, the method further comprises administering one or more additional therapeutic agent to the subject.

13. The method of claim 12, wherein the one or more additional therapeutic agent is a corticosteroid, saline, or tocilizumab.

14. The method of claim 13, wherein the corticosteroid is dexamethasone.

15. The method of claim 12, wherein the one or more additional therapeutic agent is administered in cycle 1 wherein the anti-DLL3 agent is administered.

16. The method of claim 1, wherein the platinum-based chemotherapy is platinum-etoposide therapy.

17. The method of claim 1, wherein the anti-DLL3 agent is administered by IV infusion.

18. The method of claim 1, wherein the subject is a human.
